(19) United States
(12) Patent Application Publication
(10) Pub. No.: US 2024/0075214 A1
(43) Pub. Date: Mar. 7, 2024

(54) SAFETY AND ACCURACY MEASURES FOR MEDICAMENT BOLUS DELIVERY

(71) Applicant: INSULET CORPORATION, Acton, MA (US)

(72) Inventors: Ashutosh ZADE, San Diego, CA (US); Joon Bok LEE, Acton, MA (US); Yibin ZHENG, Hartland, WI (US); Rangarajan NARAYANASWAMI, Weston, MA (US); Jason O’CONNOR, Acton, MA (US)

(21) Appl. No.: 18/450,238

(22) Filed: Aug. 15, 2023

Related U.S. Application Data
(60) Provisional application No. 63/373,258, filed on Aug. 23, 2022.

(51) Int. Cl. A61M 5/315; G16H 20/17

(52) U.S. Cl. CPC A61M 5/31566; G16H 20/17

(57) ABSTRACT

Safety and accuracy measures may be provided in a medicament delivery device. The medicament delivery device may be able to identify the inadvertent delivery of duplicate medicament boluses by a user. The exemplary embodiments may set limits for maximum allowable medicament bolus doses and maximum cumulative bolus doses over an interval. The exemplary embodiments may provide automatic meal detection and may use detection of meals in determining whether a user-requested medicament bolus dose should be adjusted or rejected. The exemplary embodiments may facilitate delivery of partial boluses and the delivery of remainders of the boluses contingent on acceptable glucose level trends of the user. The exemplary embodiments may resolve potential conflicts among multiple medicament delivery devices via a broker or by providing a single actor, such as a medicament delivery controller, that has sole control of dosing and delivery of medicament boluses.

### RELATED APPLICATIONS

This application claims the benefit of U.S. Provisional Patent Application No. 63/373,258, filed Aug. 23, 2022, the entire contents of which are incorporated herein by reference in their entirety.

### BACKGROUND

A conventional medicament delivery device may deliver boluses of medicament to a user. In some instances, there may be other medicament delivery devices that also may deliver boluses of medicament to the user. For example, the user may have an on-body medicament delivery device and a pen medicament delivery device that are both able to deliver boluses of medicament to the user. This may pose a problem if the medicament delivery devices are not synchronized and/or not aware of the complete drug delivery activity of the user. There is the possibility of over-delivery or under-delivery of medicament to the users by the multiple medicament delivery devices. A particularly troublesome possibility is where multiple medicament delivery devices deliver a medicament bolus to the user at times that are not separated by much time. For instance, where the medicament is insulin, the delivery of two insulin boluses in quick succession may pose a risk that the user is plunged into hypoglycemia by the successive insulin boluses.

Part of the challenge is that the multiple medicament delivery devices may be unaware of the medicament delivery activities of the other medicament delivery devices. As a result, the dosing and timing of the medicament boluses that they deliver may be based on incomplete or out of date information. Hence, the dose of the medicament may be inaccurate, and the timing of the delivery of the medicament bolus may be undesirable.

A further challenge is that the knowledge of data regarding the user (such as a most recent glucose level) may not be current for all the devices. Consider the example of an on-body insulin pump and an insulin pen. The on-body insulin pump may be aware of the latest glucose level reading for a user and the latest insulin on board (JOB) for the user. The insulin pen may not possess such information or may possess outdated information. Thus, the insulin pen may determine an insulin bolus dose based on outdated data, thus, increasing the risk of an insulin bolus dose that is inaccurate.

Problems also may arise where a single medicament delivery device is used to deliver medicament boluses to the user but multiple devices are involved in determining the dose of the medicament bolus. For example, an insulin pump may have the ability to determine insulin bolus doses for the user and to deliver the insulin bolus doses. A management device for the insulin pump, such as dedicated device or a smartphone with a management application, may provide a bolus calculator for the user that can be used to determine a bolus size for the user. After the insulin bolus dose is determined using the calculator, the management device may send a request to the insulin pump to deliver a bolus of the calculated dose. The insulin bolus dose sent from the management device may not be properly calculated because, for example, it may be based on an incorrect amount of insulin on board (JOB). Nevertheless, the insulin pump may respond to the request by delivering the requested insulin bolus dose to the user. The user requested insulin bolus dose also may act in combination with an insulin bolus dose recently delivered by the insulin pump to dramatically lower the glucose level of the user more than intended.

The risk of duplicate medicament bolus deliveries is not limited to an environment where there are multiple medicament delivery devices. Similar to the example above, duplicate boluses may arise where only a single medicament delivery device is in use. A user may request a medicament bolus from the medicament delivery device and then forget that a medicament bolus was requested. The user subsequently may again request the medicament bolus, having forgotten the earlier request that was delivered. The multiple medicament bolus request also may be submitted inadvertently by not being facile enough with the user interface and accidentally requesting two insulin boluses.

User requested medicament bolus doses have a heightened probability of being inaccurate. Users often miscalculate medicament bolus doses. For example, a user may request a medicament bolus that is too large. Where the medicament is insulin, a user may request an insulin bolus dose that is too large or too small for an upcoming meal. Moreover, the user may request an insulin bolus in anticipation of eating and not end up eating, or may eat at a different time than anticipated, or may eat a different amount than anticipated. The result is a risk of the user causing hypoglycemia or hyperglycemia responsive to the requested insulin bolus.

### SUMMARY

In accordance with an inventive aspect, a method is performed by a processor of an insulin delivery device. The method includes monitoring glucose level values for a user with the processor of the insulin delivery device and receiving an insulin bolus request for delivery of a specified bolus dose of insulin via the insulin delivery device to the user. The method also includes, with the processor of the insulin delivery device, determining a glucose level trend of the user based on the monitoring and adjusting the specified bolus dose of insulin in the insulin bolus request based on the determined glucose level trend of the user showing a glucose level characteristic. The method further includes delivering an insulin bolus of the adjusted specified bolus dose to the user via the insulin delivery device.

The glucose level characteristic may be a substantial rise in the glucose level of the user. The performing of the adjusting of the specified bolus dose may be performed, in some embodiments, only where the checking indicates that there is substantial rise in glucose level of the user. The adjusting may include determining a corrected insulin bolus dose to compensate for ingestion of a meal by the user. The larger of the corrected insulin bolus dose and the specified dose may be set as the adjusted specified bolus dose and delivered to the user via the insulin delivery device. The checking may determine that there is no substantial rise in glucose level of the user, deliver an insulin bolus of the specified bolus dose to the user via the insulin delivery device, not perform the adjusting of the specified bolus dose, and not deliver an insulin bolus of the adjusted specified bolus dose to the user via the insulin delivery device. The method may include displaying on a display a notification that the adjusted specified bolus dose of insulin was delivered to the user via the insulin delivery device. The method may include displaying on the display a confirmation request for confirming delivery of the adjusted specified dose.

In accordance with another inventive facet, a method is performed by a processor of an insulin delivery device. The method entails receiving a request for delivery of an insulin bolus of a specified bolus dose to a user by an insulin delivery device and determining with the processor of the insulin delivery device whether the user likely has eaten within an interval and/or whether the user has already received a bolus of insulin within a time window. Where it is determined that the user likely has eaten within the interval and/or the user has already received a bolus of insulin within the time window, the method includes adjusting the specified bolus dose of the requested insulin bolus with the processor of the insulin delivery device to account for the user likely having eaten and/or the user having already received the bolus of insulin in the time window. The method additionally includes, with the processor of the insulin delivery device, causing delivery of the insulin bolus of the adjusted specified bolus dose to the user by the insulin delivery device.

The request may be from an external electronic device. The external electronic device may be a management device for the insulin delivery device. The determining whether the user likely has eaten within an interval may include analyzing a glucose level trend of the user. The analyzing of the glucose level trend of the user may entail analyzing the glucose level trend to identify a rise in glucose level associated with ingesting a meal. The analyzing of the glucose level may be performed by at least one machine learning model. The analyzing of the glucose level may be performed by multiple machine learning models. It may be determined that the user likely has eaten within the interval, and the adjusting of the dose of the requested insulin bolus may include increasing the dose of the requested insulin bolus. It may be determined that the user likely has not eaten within the interval, and the adjusting of the dose of the requested insulin bolus may entail decreasing the dose of the requested insulin bolus. It may be determined that the user has already received a bolus of insulin within the time window, and the adjusting of the dose of the requested insulin bolus may include decreasing the dose of the requested insulin bolus. It may be determined that the user is not likely to have eaten within the interval, and the adjusting the dose of the requested insulin bolus with the processor of the insulin delivery device may include adjusting the dose of the requested insulin bolus to zero. It may be determined that the user has already received a bolus of insulin within the time window, and the adjusting of the dose of the requested insulin bolus with the processor of the insulin delivery device may entail adjusting the dose of the requested insulin bolus to zero.

In accordance with another inventive facet, an insulin delivery device for delivering insulin to a user includes a reservoir for storing insulin and a non-transitory computer-readable storage medium for storing computer programming instructions for a single insulin delivery controller that is in control of determining doses and causing delivery of insulin to the user from the insulin delivery device to the user. The single insulin delivery controller includes a bolus calculator for calculating doses for insulin boluses to be delivered to the user and an automated insulin delivery controller for controlling automated insulin delivery to the user. The insulin delivery device further includes a processor for executing the computer programming instructions of the single insulin delivery controller to determine bolus doses and to cause delivery of insulin boluses to the user from the insulin delivery device.

### BRIEF DESCRIPTION OF THE DRAWINGS

FIG. 1 depicts an illustrative medicament delivery system suitable for the exemplary embodiments.

FIG. 2 depicts a flowchart of illustrative steps that may be performed in exemplary embodiments to prevent delivery of excessive bolus doses when the medicament is insulin.

FIG. 3 depicts a flowchart of illustrative steps that may be performed in exemplary embodiments to determine what dose of insulin bolus to deliver to the user response to an insulin bolus request from the user.

FIG. 4A depicts a flowchart of illustrative steps that may be performed in exemplary embodiments to obtain meal signals.

FIG. 4B depicts an illustrative sliding window of an exemplary embodiment that encompasses glucose level values of the user over an exemplary two-hour time period.

FIG. 5 depicts a flowchart of illustrative steps that may be performed in exemplary embodiments to properly train a classifier.

FIG. 6 depicts a flowchart of illustrative steps that may be performed in exemplary embodiments to limit the insulin bolus deliveries to the user over a period.

FIG. 7 depicts an illustrative table that may be used in exemplary embodiments to specify the maximum allowable cumulative bolus doses over time periods.

FIG. 8 depicts an illustrative warning that may be sent to the user in exemplary embodiments to inform the user that the requested insulin bolus dose exceeds a maximum allowable bolus dose limit.

FIG. 9 depicts a flowchart of illustrative steps that may be performed in exemplary embodiments to identify duplicate boluses of insulin.

FIG. 10 depicts an illustrative user interface in exemplary embodiments for gathering information regarding potentially duplicate boluses.

FIG. 11 depicts an illustrative user interface element for flagging to the user that the insulin boluses appear to be duplicates and for getting confirmation from the user.

FIG. 12 depicts a flowchart of illustrative steps that may be performed in exemplary embodiments to determine an insulin bolus dose based on glucose trend.

FIG. 13 depicts a flowchart of illustrative steps that may be performed in exemplary embodiments in delivering a partial insulin bolus dose followed by the remainder with a potential termination of delivery of the remainder.

FIG. 14 depicts a flowchart of illustrative steps that may be performed in exemplary embodiments to determine the dose of the partial bolus that is delivered initially.

FIG. 15 depicts a flowchart of illustrative steps that may be performed relative to provisional bolus doses.

FIG. 16 depicts an illustrative user interface of an exemplary embodiment for choosing between a confident bolus dose and a provisional bolus dose.

FIG. 17 depicts an illustrative user interface of an exemplary embodiment for choosing to accept or reject delivery of a provisional bolus dose.

FIG. 18 depicts an illustrative user interface of an exemplary embodiment for entering information regarding a medicament bolus dose delivered by an auxiliary device.

FIG. 19 depicts an illustrative message that may be sent to a medicament delivery device from another device to provide information regarding the auxiliary bolus in exemplary embodiments.

FIG. 20 depicts a flowchart of illustrative steps that may be performed in exemplary embodiments to use a broker to perform reconciliation of requests for medicament bolus delivery.

FIG. 21 depicts an illustrative table of conditions and actions that are taken responsive to the conditions to determine the final dose from recommendations in exemplary embodiments.

FIG. 22A depicts a format of an illustrative message that may be sent from a medicament delivery device to a management device in order to facilitate the sharing of information in exemplary embodiments.

FIG. 22B depicts the format of an illustrative message that may be sent from the management device to the medicament delivery device in exemplary embodiments.

FIG. 23A depicts a conventional arrangement wherein there is an actor affecting medicament bolus delivery on a medicament delivery device and another actor affecting medicament bolus delivery on the management device.

FIG. 23B depicts an illustrative arrangement suitable for exemplary embodiments where there is only a single actor affecting medicament bolus delivery.

### DETAILED DESCRIPTION

Exemplary embodiments attempt to address the above-discussed problems associated with medicament bolus deliveries. The exemplary embodiments may set a maximum allowed dose for a medicament, like insulin. The exemplary embodiments also may set limits for cumulative medicament bolus doses over time periods. These limits help to prevent a user from delivering an excessive amount of medicament via bolus. The exemplary embodiments may detect meal ingestion for the user by analyzing glucose level trends for the user and adjust the maximum allowed medicament bolus dose or cumulative dose over a time window. The exemplary embodiments may also modify or reject medicament bolus doses requested by the user based on factors, such as recent blood glucose level reading and insulin on board (JOB). Further, the exemplary embodiments may identify duplicate medicament bolus doses that were delivered by request of the user. The user may be informed of such duplicate medicament bolus deliveries.

Partial medicament bolus doses may be delivered initially in response to a request. The partial medicament bolus dose delivery may be followed by slower delivery of the remainder with glucose trend monitoring. The delivery of the remainder may be terminated based on a glucose level trend, such as if the glucose level trend turns from positive to negative. In some exemplary embodiments, the user may have the option of choosing to deliver the whole medicament bolus dose immediately or instead to deliver a partial bolus followed by contingent delivery of the remainder of the whole medicament bolus dose based on the results of glucose monitoring.

The exemplary embodiments may accommodate multiple medicament delivery devices and may ensure that the actions of the multiple medicament delivery devices do not result in poor medicament bolus dosing and/or timing of delivery. The exemplary embodiments may provide a mechanism, such as a user interface, for information regarding medicament bolus doses delivered by auxiliary medicament delivery devices to be recorded and passed to other medicament delivery devices. Thus, knowledge of the actions of other actors (i.e., other medicament delivery devices that may deliver medicament to the user) is known.

The exemplary embodiments may provide a “broker” on a primary medicament delivery device for reconciling recommendations for delivery of medicament bolus doses from a secondary medicament delivery device with those of the primary medicament delivery device. Based on the state of knowledge of the secondary medicament delivery device and timing of receipt of recommendations received in temporal proximity (close in time to each other), the broker may decide what the final medicament bolus dose should be and trigger delivery of the final medicament bolus dose by the primary medicament delivery device.

The exemplary embodiments may provide a single actor that decides medicament bolus doses and triggers medicament bolus dose delivery. The use of a single actor eliminates conflicts among multiple medicament delivery devices. The single actor has complete and current knowledge of the requisite information needed to determine medicament bolus doses and to trigger delivery of medicament bolus doses to the user.

It should be appreciated that in the discussion below, some of the embodiments and discussed features apply broadly to medicament delivery devices, whereas other embodiments apply to more narrowly to insulin delivery devices. The discussion below attempts to clarify whether the embodiments or discussed features apply to medicament delivery devices or solely to insulin delivery devices.

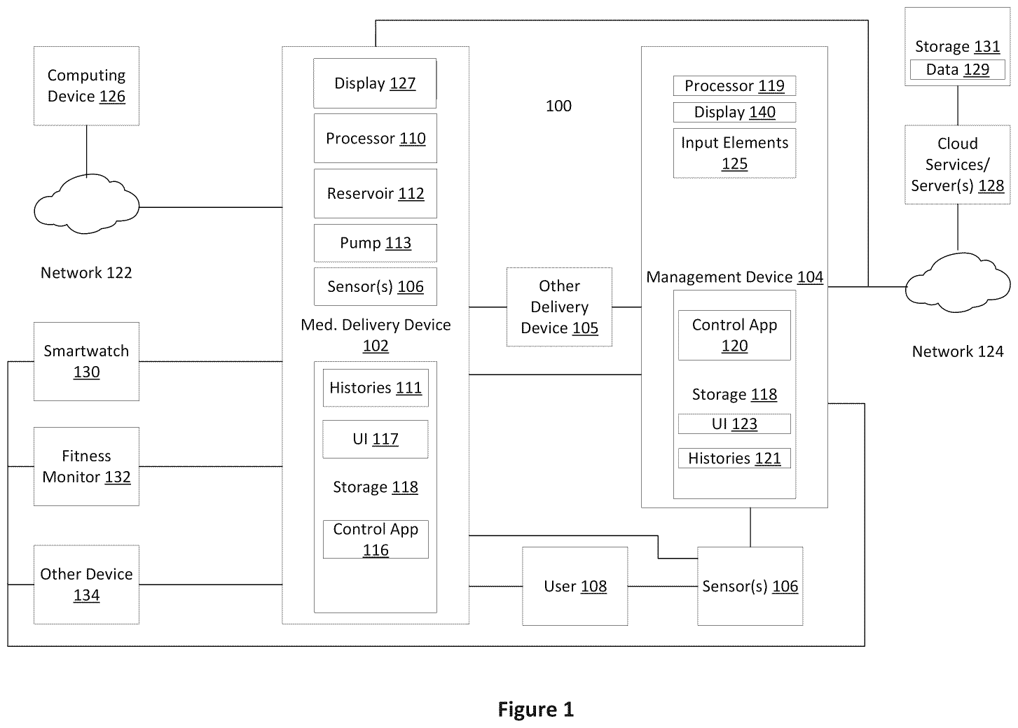

FIG. 1 depicts an illustrative medicament delivery system 100 that is suitable for delivering a medicament to a user 108 in accordance with the exemplary embodiments. The medicament delivery system 100 includes a medicament delivery device 102. The medicament delivery device 102 may be a wearable device that is worn on the body of the user 108 or carried by the user. The medicament delivery device 102 may be directly coupled to a user (e.g., directly attached to a body part and/or skin of the user 108 via an adhesive or the like) with no tubes and an infusion location directly under the medicament delivery device 102, or carried by the user (e.g., on a belt or in a pocket) with the medicament delivery device 102 connected to an infusion site where the medicament is injected using a needle and/or cannula. In a preferred embodiment, a surface of the medicament delivery device 102 may include an adhesive to facilitate attachment to the user 108.

The medicament delivery device 102 may include a processor 110. The processor 110 may be, for example, a microprocessor, a logic circuit, a field programmable gate array (FPGA), an application specific integrated circuit (ASIC) or a microcontroller. The processor 110 may maintain a date and time as well as other functions (e.g., calculations or the like). The processor 110 may be operable to execute a control application 116 encoded in computer programming instructions stored in the storage 114 that enables the processor 110 to direct operation of the medicament delivery device 102. The control application 116 may be a single program, multiple programs, modules, libraries or the like. The processor 110 also may execute computer programming instructions stored in the storage 114 for a user interface (UI) 117 that may include one or more display screens shown on display 127. The display 127 may display information to the user 108 and, in some instances, may receive input from the user 108, such as when the display 127 is a touchscreen.

The control application 116 may control delivery of a medicament to the user 108 per a control approach like that described herein. The control application 116 may provide the safety and accuracy measures relating to medicament boluses that are described herein. The storage 114 may hold histories 111 for a user, such as a history of basal deliveries, a history of bolus deliveries, and/or other histories, such as a meal event history, exercise event history, glucose level history, and/or the like. In addition, the processor 110 may be operable to receive data or information. The storage 114 may include both primary memory and secondary memory. The storage 114 may include random access memory (RAM), read only memory (ROM), optical storage, magnetic storage, removable storage media, solid state storage or the like.

The medicament delivery device 102 may include one or more housings for housing its various components including a pump 113, a power source (not shown), and a reservoir 112 for storing a medicament for delivery to the user 108. A fluid path to the user 108 may be provided, and the medicament delivery device 102 may expel the medicament from the reservoir 112 to deliver the medicament to the user 108 using the pump 113 via the fluid path. The fluid path may, for example, include tubing coupling the medicament delivery device 102 to the user 108 (e.g., tubing coupling a cannula to the reservoir 112), and may include a conduit to a separate infusion site. The medicament delivery device may have operational cycles, such as every 5 minutes, in which basal doses of medicament are calculated and delivered as needed. These steps are repeated for each cycle.

There may be one or more communications links with one or more devices physically separated from the medicament delivery device 102 including, for example, a management device 104 of the user and/or a caregiver of the user, sensor(s) 106, a smartwatch 130, a fitness monitor 132 and/or another variety of device 134. The communication links may include any wired or wireless communication links operating according to any known communications protocol or standard, such as Bluetooth®, Wi-Fi, a near-field communication standard, a cellular standard, or any other wireless protocol.

The medicament delivery device 102 may interface with a network 122 via a wired or wireless communications link. The network 122 may include a local area network (LAN), a wide area network (WAN) or a combination therein. A computing device 126 may be interfaced with the network 122, and the computing device may communicate with the medicament delivery device 102.

The medicament delivery system 100 may include one or more sensor(s) 106 for sensing the levels of one or more analytes. The sensor(s) 106 may be coupled to the user 108 by, for example, adhesive or the like and may provide information or data on one or more medical conditions and/or physical attributes of the user 108. The sensor(s) 106 may be physically separate from the medicament delivery device 102 or may be an integrated component thereof. The sensor(s) 106 may include, for example, glucose monitors, such as continuous glucose monitors (CGM's) and/or non-invasive glucose monitors. The sensor(s) 106 may include ketone sensors, analyte sensors, heart rate monitors, breathing rate monitors, motion sensors, temperature sensors, perspiration sensors, blood pressure sensors, alcohol sensors, or the like.

The medicament delivery system 100 may or may not also include a management device 104. In some embodiments, no management device is needed as the medicament delivery device 102 may manage itself. The management device 104 may be a special purpose device, such as a dedicated personal diabetes manager (PDM) device. The management device 104 may be a programmed general-purpose device, such as any portable electronic device including, for example, a dedicated controller, such as a processor, a micro-controller, or the like. The management device 104 may be used to program or adjust operation of the medicament delivery device 102 and/or the sensor(s) 106. The management device 104 may be any portable electronic device including, for example, a dedicated device, a smartphone, a smartwatch, or a tablet. In the depicted example, the management device 104 may include a processor 119 and a storage 118. The processor 119 may execute processes to manage a user's glucose levels and to control the delivery of the medicament to the user 108. The medicament delivery device 102 may provide data from the sensors 106 and other data to the management device 104. The data may be stored in the storage 118. The processor 119 may also be operable to execute programming code stored in the storage 118. For example, the storage 118 may be operable to store one or more control applications 120 for execution by the processor 119. The control application 120 may be responsible for controlling the medicament delivery device 102, such as by controlling the automated insulin delivery (AID) of insulin to the user 108. In some exemplary embodiments, the control application 120 provides the adaptability described herein. The storage 118 may store the control application 120, histories 121 like those described above for the medicament delivery device 102, and other data and/or programs.

A display 140, such as a touchscreen, may be provided for displaying information. The display 140 may display user interface (UI) 123. The display 140 also may be used to receive input, such as when it is a touchscreen. The management device 104 may further include input elements 125, such as a keyboard, button, knobs, or the like, for receiving input form the user 108.

The management device 104 may interface with a network 124, such as a LAN or WAN or combination of such networks, via wired or wireless communication links. The management device 104 may communicate over network 124 with one or more servers or cloud services 128. Data, such as sensor values, may be sent, in some embodiments, for storage and processing from the medicament delivery device 102 directly to the cloud services/server(s) 128 or instead from the management device 104 to the cloud services/server(s) 128.

Other devices, like smartwatch 130, fitness monitor 132 and device 134 may be part of the medicament delivery system 100. These devices 130, 132 and 134 may communicate with the medicament delivery device 102 and/or management device 104 to receive information and/or issue commands to the medicament delivery device 102. These devices 130, 132 and 134 may execute computer programming instructions to perform some of the control functions otherwise performed by processor 110 or processor 119, such as via control applications 116 and 120. These devices 130, 132 and 134 may include displays for displaying information. The displays may show a user interface for providing input by the user, such as to request a change or pause in dosage, or to request, initiate, or confirm delivery of a bolus of a medicament, or for displaying output, such as a change in dosage (e.g., of a basal delivery amount) as determined by processor 110 or management device 104. These devices 130, 132 and 134 may also have wireless communication connections with the sensor 106 to directly receive analyte measurement data. Another delivery device 105, such as a medicament delivery pen, may be accounted for or may be provided for also delivering medicament to the user 108. The other device 105 is referred to herein as a secondary medicament delivery device or auxiliary device, whereas the medicament delivery device 102 is referred to as the primary medicament delivery device.

A wide variety of medicaments may be delivered by the medicament delivery device 102 and delivery device 105. The medicament may be insulin for treating diabetes. The medicament may be glucagon for raising a user's glucose level. The medicament may also be a glucagon-like peptide (GLP)-1 receptor agonists for lowering glucose or slowing gastric emptying, thereby delaying spikes in glucose after a meal. Alternatively, the medicament delivered by the medicament delivery device 102 may be one of a pain relief agent, a chemotherapy agent, an antibiotic, a blood thinning agent, a hormone, a blood pressure lowering agent, an antidepressant, an antipsychotic, a statin, an anticoagulant, an anticonvulsant, an antihistamine, an anti-inflammatory, a steroid, an immunosuppressive agent, an antianxiety agent, an antiviral agent, a nutritional supplement or a vitamin. The medicament may be a coformulation of two or more of those medicaments listed above.

The functionality described herein for the exemplary embodiments may be under the control of or performed by the control application 116 of the medicament delivery device 102 or the control application 120 of the management device 104. In some embodiments, the functionality wholly or partially may be under the control of or performed by the cloud services/servers 128, the computing device 126 or by the other enumerated devices, including smartwatch 130, fitness monitor 132 or another wearable device 134.

In the closed loop mode, the control application 116, 120 determines the medicant delivery amount for the user 108 on an ongoing basis based on a feedback loop. For an insulin delivery device, the aim of the closed loop mode is to have the user's glucose level at a target glucose level or within a target glucose range.

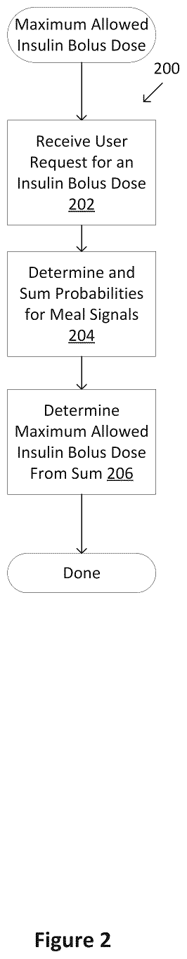

As was mentioned above, the exemplary embodiments provide a number of safety measures to protect the user 108. One of those features is particular to the instance where the medicament is insulin and this feature limits boluses of insulin to a maximum allowed insulin bolus dose to prevent excessive delivery of insulin to the user, regardless of whether the insulin is from device 102 or device 105. FIG. 2 depicts a flowchart 200 of illustrative steps that may be performed in exemplary embodiments to prevent delivery of excessive bolus doses of insulin. At 202, a request for an insulin bolus of a specified dose is received by the control application 116 of the medicament delivery device 102. The user 108 may enter such a request via the user interface 117 of the medicament delivery device or alternatively, via a user interface 123 of the management device 104. In some alternative embodiments, the user 108 may enter the insulin bolus request via one of devices 130, 132 or 134.

The control application 116 of the exemplary embodiments may process the insulin bolus request to see if the insulin bolus dose that is requested exceeds a maximum allowed insulin bolus dose. The magnitude of the maximum allowed insulin bolus dose depends on whether the user 108 has eaten or not. When a meal is detected, it is safer for the user 108 to bolus a larger dose because there is a reduced likelihood of a hypoglycemic event for the user 108. To that end, the exemplary embodiments attempt to determine if the user has eaten. The exemplary embodiments may analyze meal signals that represent a probability that the user has eaten. The meal signals and how the signals are determined are discussed below. At 204, the probabilities of a meal generated by the meal signals are determined, and the probabilities for all of the meal signals are summed. For instance, if three meal signals are used and the respective probabilities of a meal associated with the meal signals are 0.2, 0.3 and 0.5, the sum of the probabilities for the meal signals would be 1, indicating a 100% probability that a meal has been detected.

At 206, the maximum allowed insulin bolus dose may be determined from the sum of the probabilities of the meal signals. In particular, the maximum allowed insulin bolus may be determined as:

Maximum Allowed Insulin Bolus Dose=X′*(1-X) (Eq. 1)

where X is the sum of probabilities of the meal signals and X′=((TDI/2)*0.05), where TDI is the total daily insulin for the user 108. The value of 0.05 is a safety factor that is configurable The value TDI/2 represents an ideal portion of TDI that is dedicated to insulin boluses per day, and the value 0.05, limits the max allowed bolus size to 5% of that ideal portion. From Equation 1, if the sum of the probabilities (X) is 1 (indicating that a meal has been detected), the maximum allowed bolus dose is 0; whereas if the sum of the probabilities is 0 (indicating that no meal has been detected), the maximum allowed bolus dose is X′. For cases where the sum of probabilities is in between 0 and 1, the maximum allowed bolus dose is a reduced portion of X′, with greater reduction as the sum of probabilities approaches 1, indicating an increased probability of a meal.

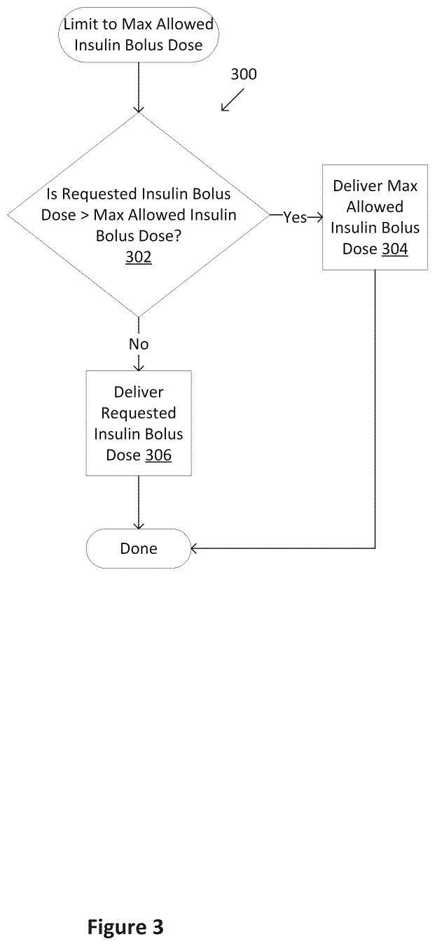

FIG. 3 depicts a flowchart 300 of illustrative steps that may be performed in exemplary embodiments to determine what dose of insulin bolus to deliver to the user 108 in response to an insulin bolus request from the user 108. These steps may be performed by the control application running on the processor 110. At 302, a check is made whether the requested insulin bolus dose requested is greater than the maximum allowed insulin bolus dose that was determined (see 206). At 304, if the requested insulin bolus dose exceeds the maximum allowed insulin bolus dose, the maximum allowed insulin bolus dose is delivered. If not, at 306, the user requested insulin bolus dose is delivered. Another way of viewing this approach is that the minimum of the maximum allowed insulin bolus dose and the user requested insulin bolus dose is selected.

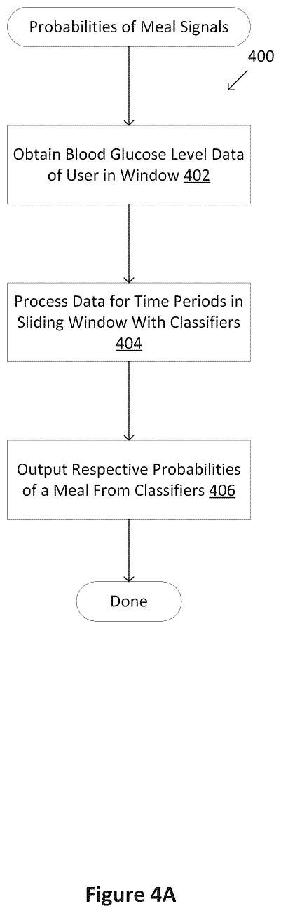

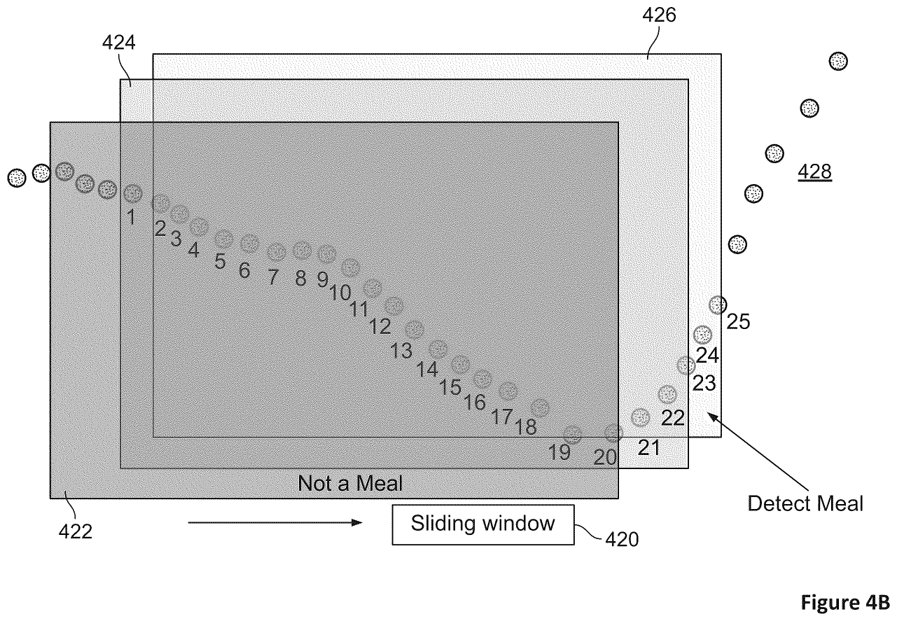

The meal signals represent probabilities that a meal has been detected in blood glucose level data for the user 108 at successive time intervals. FIG. 4A depicts a flowchart 400 of illustrative steps that may be performed in exemplary embodiments to obtain the meal signals. In accordance with this approach, glucose rises of specified or predetermined magnitudes within respective time periods are sought. For example, the approach may look for instances of a 20 mg/dL rise or greater within a 15-minute period, a 40 mg/dL rise or greater in a 30-minute period and a 60 mg/dL rise or greater in a 60-minute period. At 402, blood glucose level data of the user 108 is obtained for 15-minute periods, 30-minute periods and 60-minute periods within a window, such as a two-hour window, ending with a most recent glucose level reading. Hence, the rises described above within a 15-minute window are examples of one such signal.

In the exemplary embodiments, at 404, classifiers process the glucose level data to identify glucose level rises for respective time periods, such as 15 minutes, 30 minutes and 60 minutes. A separate classifier may be provided for each time period. The classifiers may be machine learning models that recognize patterns of glucose level rises indicative of meals. Thus, one classifier may look for glucose level rises over 15-minute intervals in the window. Another classifier may look for glucose level rises over 30-minute intervals in the window, and a third classifier may look for glucose level rises over 60-minute intervals in the window. Each classifier may be, for example, a separate neural network model or a separate decision tree model. The classifiers may output the probability that a meal has been detected within the window based on what glucose level rise is detected, if any.

At 406, the classifiers output their respective probabilities, which are used in determining probabilities of whether there has been a meal event, and in determining the maximum allowed insulin bolus dose, as described above. The monitoring of the glucose level data of the user 108 may be performed on an ongoing basis.

FIG. 4B depicts an example sliding window 420 that slides over time to encompass an updated two-hour period as new glucose level readings are received. The sliding window 420 starts off in position or window 422 that covers glucose level readings 428 up to reading 20. In a second position or window 424, the window encompasses readings 428 from reading 1 to reading 22, and in the third position or window 426, the window encompasses from reading 2 to 25. Each reading may occur at 5-minute intervals; thus, one window may encompass a two-hour period. In positions or windows 422 and 424, no meal is detected, but a meal is detected in position or window 426 based on the glucose level rise.

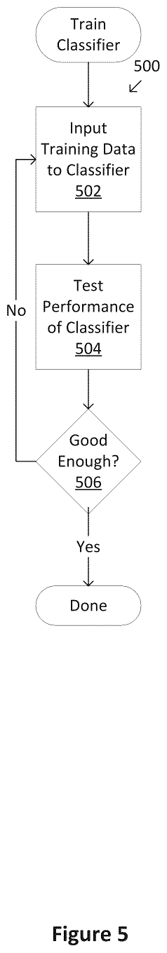

The classifiers need to be trained before they are used in detecting meal signals. FIG. 5 depicts a flowchart 500 of illustrative steps that may be performed in exemplary embodiments to properly train a classifier. Each of the classifiers may be trained in this fashion. To train a classifier, at 502, input data, such as past, glucose level data where the meal events are known is input to the classifier. Preferably, a large amount of input data is used in training to ensure that the classifier is sufficiently trained. The classifier adapts based on the training data to identify glucose level rises associated with a user eating a meal. The classifier learns what the probability is that a given glucose rise pattern indicates that the user has eaten a meal.

After a period of training has been performed, at 504, the performance of the classifier may be checked. In other words, a check is made of how well the classifier predicts the probability of a meal from glucose level data in its current state of learning. At 506, a check is made whether the classifier performs well enough. This may entail determining whether the probabilities of a meal output by the classifier are accurate enough relative the actual consumption of meals by the user, which is known for the training data. An accuracy threshold of the predictability of the meal by the classifier may be established, and if the classifier predicts the meal outputs above the threshold, training may be completed. Otherwise, more training is needed and the process repeats at 502 with additional training data.

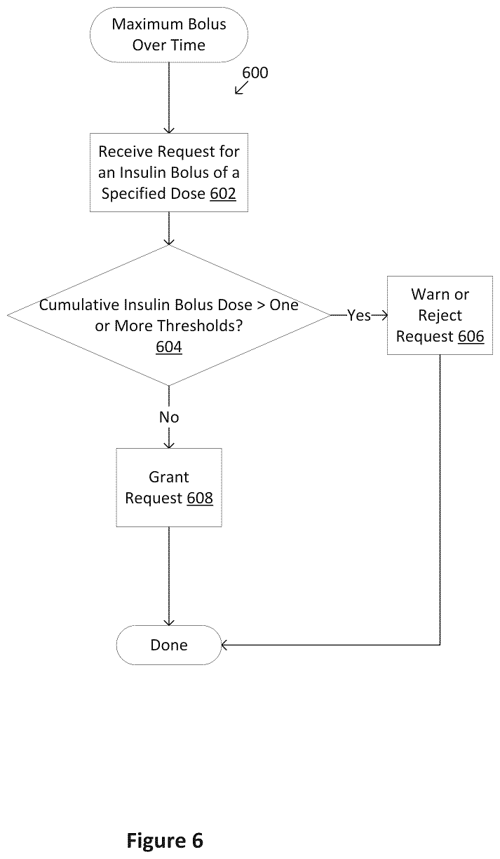

Another safety feature that may be provided in exemplary embodiments is to limit the cumulative amount of medicament bolus over time. Such a limit prevents excessive bolus doses that may heighten the risk of an undesired outcome. For instance, where the medicament is insulin, the risk of hypoglycemia may increase with greater medicament bolus doses. FIG. 6 depicts a flowchart 600 of illustrative steps that may be performed in exemplary embodiments to limit the medicament bolus deliveries to the user 108 over a period where the medicament is insulin. At 602, a request for an insulin bolus dose is received. At 604, a check is made whether the cumulative insulin boluses over one or more time periods are not exceeded if an insulin bolus of the requested dose is delivered to the user 108.

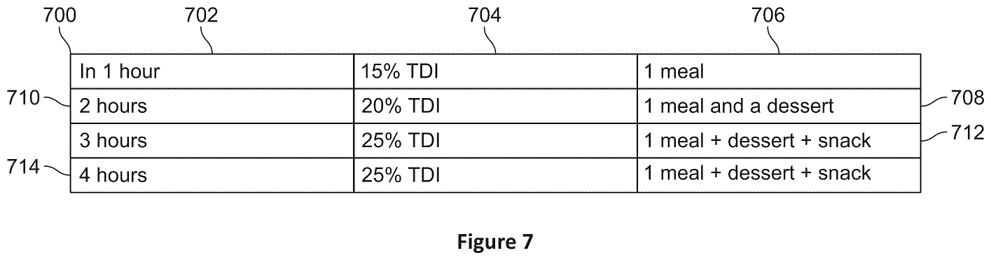

FIG. 7 depicts an illustrative table 700 that may be used in exemplary embodiments to specify the maximum cumulative bolus dose of insulin to a user over different time periods. Column 702 specifies time periods, and column 704 specifies the thresholds of the maximum insulin bolus permitted for the respective time periods. Column 706 specifies an illustration of what type of eating (e.g., a meal or a meal plus a dessert) is associated with the threshold. Each row is associated with a time period. Thus, row 708 is associated with a one-hour period, row 710 is associated with a two-hour period, row 712 is associated with a three-hour period, and row 714 is associated with a four-hour period. When this table 700 is used, at 604, a check is made that the cumulative insulin bolus dose to the user 108 (which includes the requested dose) does not exceed 15% of the TDI for the user 108 in an hour period. In addition, a check is made that the cumulative insulin bolus dose over the past two hours does not exceed 20% of the TDI for the user, the cumulative insulin bolus dose over the past three hours does not exceed 25% of the TDI for the user, and the cumulative insulin bolus dose over the past four hours does not exceed 25% of the TDI for the user.

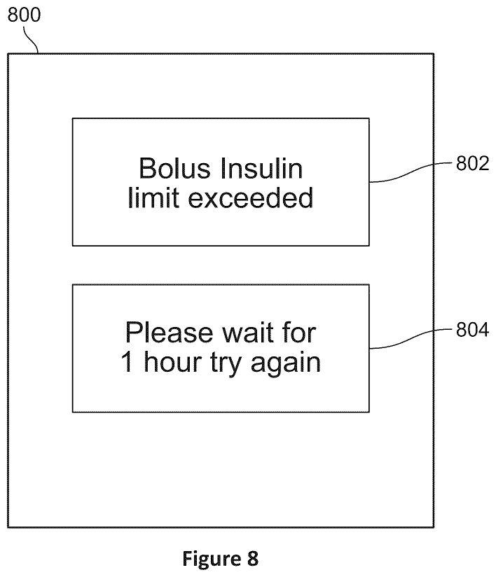

If the cumulative insulin bolus dose is over one or more of the thresholds for the respective time periods, at 606, a warning is sent to the user 108 or the insulin bolus request is rejected. FIG. 8 depicts an example of such a warning 800 that may be displayed in exemplary embodiments to inform the user 108 that the requested insulin bolus dose exceeds a limit. The warning 800 may be displayed as part of the user interface 117 on display 127 of the medicament delivery device 102 or as part of the user interface 123 on display 140. The warning 800 may include an explanation 802 of what went wrong (e.g., “Bolus Insulin limit exceeded”). The warning may also include guidance 804 (e.g., “Please wait for 1 hour and try again”). A different message may also be generated if the response is an outright rejection of the bolus request.

If the cumulative bolus dose for the time periods is not exceeded, at 608, the request is granted so that the medicament delivery device 102 delivers the requested insulin bolus to the user 108.

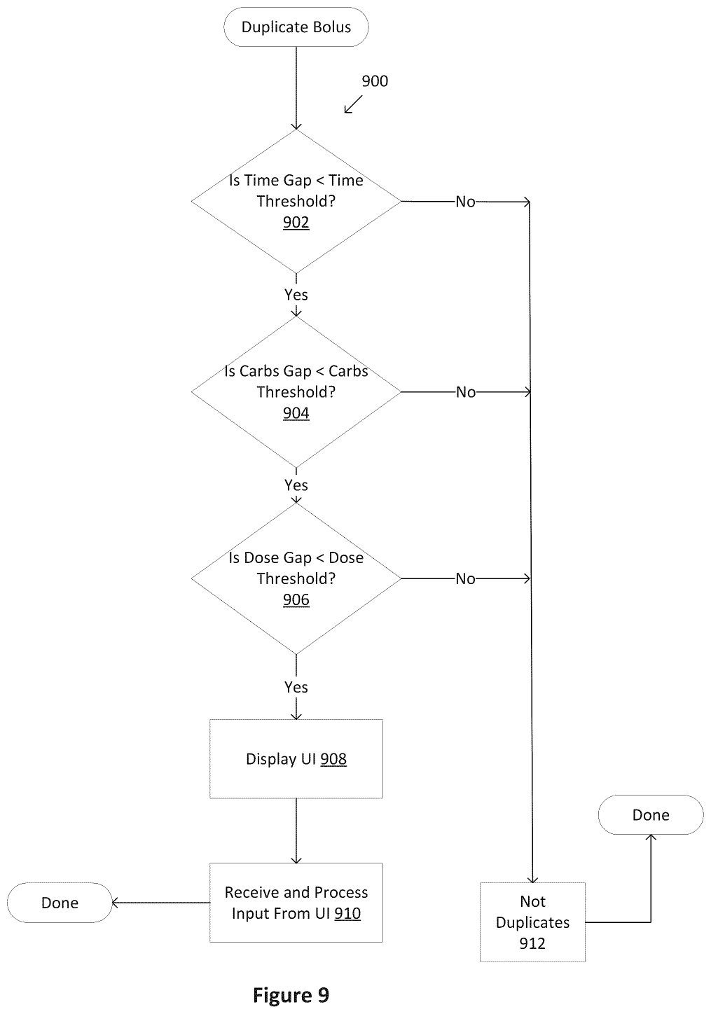

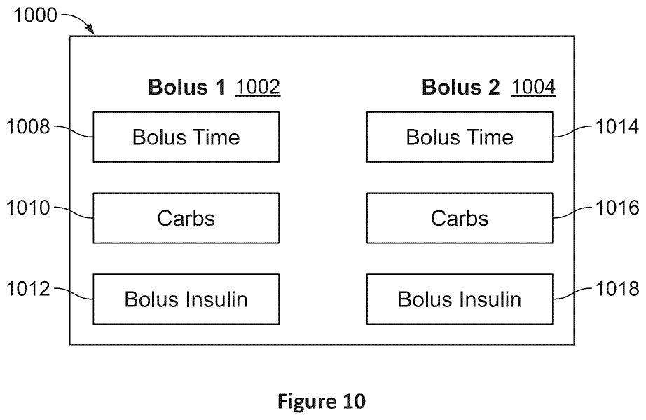

Another safeguard that may be provided in exemplary embodiments is to identify duplicate insulin boluses. FIG. 9 depicts a flowchart of illustrative steps that may be performed in exemplary embodiments to identify such duplicate insulin boluses. If two insulin boluses are delivered within a configurable time period, such as within 30 minutes of each other, the steps of FIG. 9 may be performed. As shown in an example 1000 in FIG. 10, information 1002 regarding bolus 1 and information 1004 regarding bolus 2 are known and may be compared to identify whether the boluses are duplicates. The information for each bolus may include bolus time, 1008, 1014, the carbohydrate amounts of the meal 1010, 1016 for which the boluses were delivered, and the bolus insulin doses 1012, 1018. At 902, the times 1008 and 1014 that the boluses were delivered are compared to see if they are with a threshold time amount relative to each other. If the boluses are duplicates, the time difference should not be large. If the time difference of the boluses exceeds the threshold, at 912, it is determined that that the insulin boluses are not duplicates. If the time gap between the boluses is less than the threshold, the boluses may be duplicates and the comparison continues. At 904, the carbohydrate amounts 1010 and 1016 are compared to a threshold. If the carbohydrate amounts differ by more than the threshold, at 912, the insulin boluses are determined to not be duplicates and no further action is taken. If the difference in carbohydrate amounts 1010 and 1016 is less than the threshold, at 906, the bolus insulin doses 1012 and 1018 are compared to determine whether they differ by less than a threshold. If the difference in bolus doses is greater than the threshold, at 912, the boluses are deemed to not be duplicates.

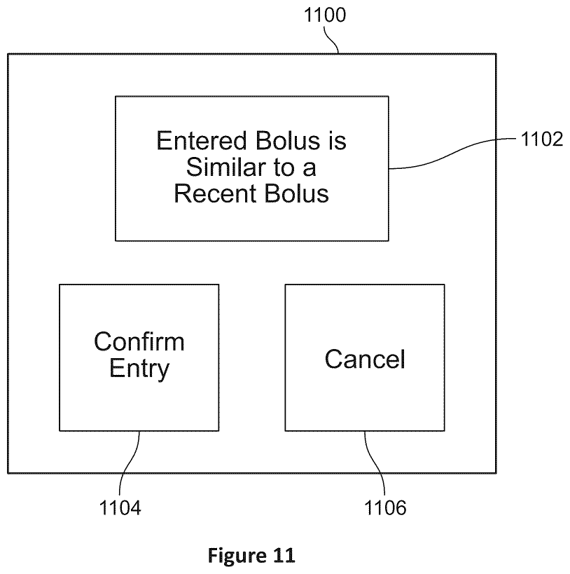

If the difference is less than the threshold, it is concluded that the insulin boluses likely are duplicates. Where the insulin boluses are likely be duplicates, at 908, a user interface may be displayed to notify the user 108 of the possible duplicate boluses and/or to get confirmation from the user 108. FIG. 11 depicts an illustrative user interface element 1100 for flagging to the user that the insulin boluses appear to be duplicates and to get confirmation. The user interface element 1100 may contain a notification 1102 that the boluses appear similar. The user interface element 1100 may contain buttons or other elements for providing input. Button 1104 may be selected to confirm that the boluses were not duplicates, and button 1106 may be selected to acknowledge that the boluses were unintended duplicates and the most recent entry should be cancelled. The user interface 1100 helps the user 108 be aware of the duplicate boluses so that the user can respond, such as by eating a snack or meal to bolster their glucose level.

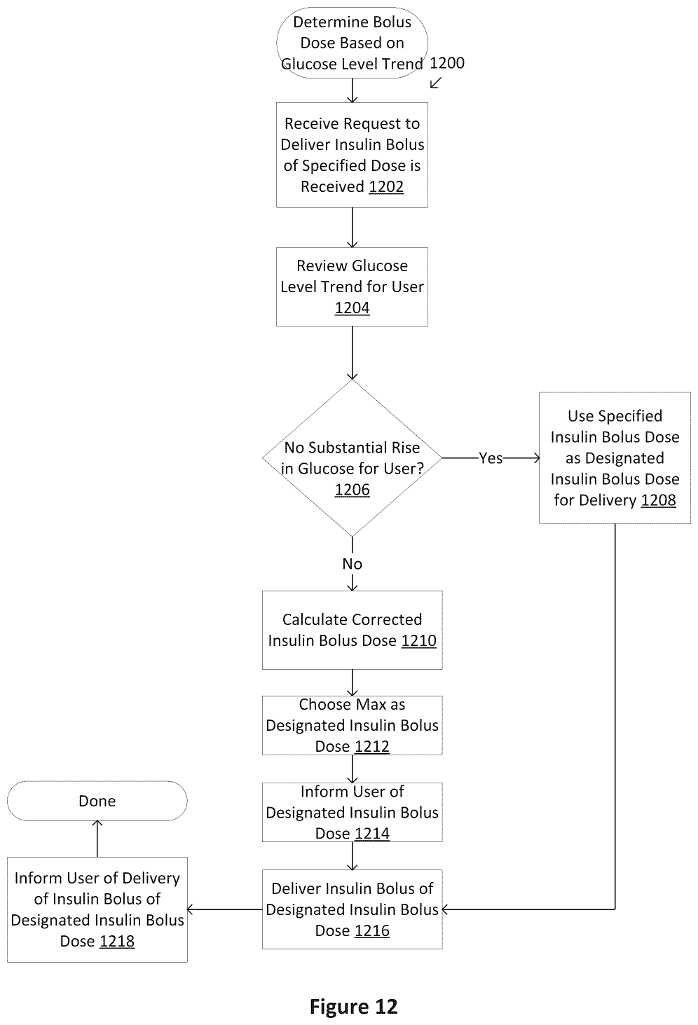

The exemplary embodiments may calculate an insulin bolus dose for the user 108 based on a current glucose level trend to provide the user with an insulin bolus dose tailored to the user's current needs. A flowchart 1200 of illustrative steps that may be performed in exemplary embodiments to determine a bolus dose based on a current glucose level trend is shown in FIG. 12. At 1202, a request to deliver an insulin bolus of a specified dose to the user 108 is received. The request may originate from the user 108. At 1204, the glucose level trend of the user 108 is reviewed. At 1206, a check is made whether there is no substantial rise in glucose for the user 108. Such a substantial rise is indicative of a meal having been ingested by the user 108. What constitutes a substantial rise may be specified by a threshold amount over a time period and may be tailored for the user 108 based on past glucose level data for the suer 108. If there is a substantial rise, the dose in the request is used as the bolus is likely for a meal, and the user is in the best position to determine the size of the meal and the proper dose for compensating for the rise in glucose that would result from ingesting the meal. Thus, at 1208, in that case, the specified insulin bolus dose is specified as the designated insulin bolus dose for delivery by the medicament delivery device 102. However, if there is not a substantial rise in the glucose indicative of a meal, at 1210, a correction insulin bolus dose is calculated based on the most recent glucose level value of the user 108. At 1212, the maximum of the user request insulin bolus dose and the corrected insulin bolus dose is chosen as the insulin bolus dose to be delivered (i.e., as the “designated insulin bolus dose”). At 1214, the user is informed of the insulin bolus dose to delivered. The notice may be a textual message displayed on a user interface, such as 117 or 123. At 1216, the determined insulin bolus dose is delivered by the medicament delivery device 102. At 1218, the user 108 is informed of the delivery of the determined insulin bolus dose.

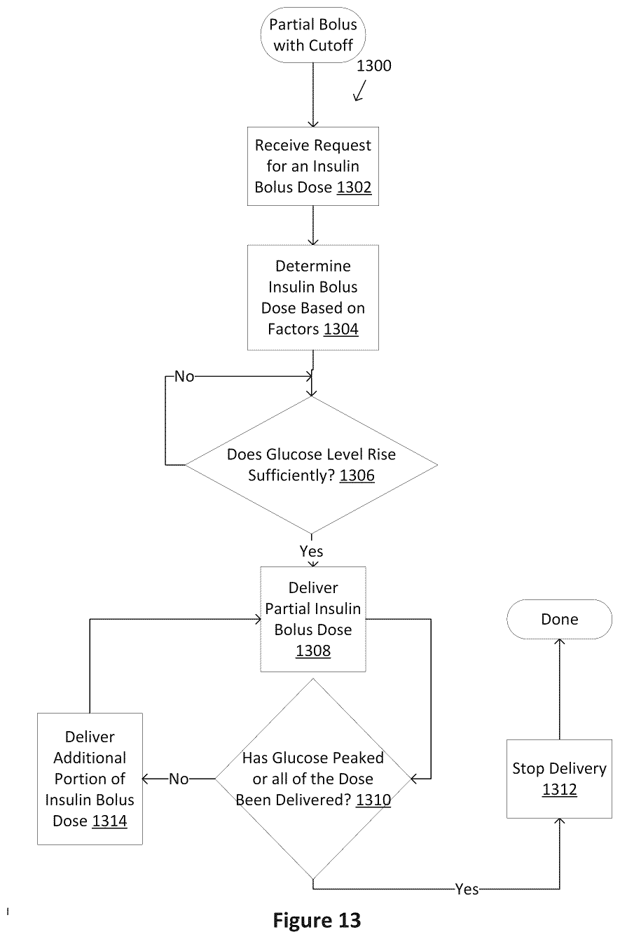

An additional safety approach that may be provided in exemplary embodiments is to only deliver a portion of the insulin bolus dose that was requested by the user 108 and then monitor the glucose trend of the user 108 to determine whether to deliver the remaining portion of the insulin bolus dose. FIG. 13 depicts a flowchart 1300 of illustrative steps that may be performed in exemplary embodiments in delivering a bolus in accordance with this approach. At 1302, a user request for an insulin bolus dose is received. At 1304, an insulin bolus dose is determined based on a number of factors (e.g., current blood glucose level, JOB, etc.) by the medicament delivery device 102. At 1306, a check is made whether the glucose level of the user 108 rises sufficiently or not. This check helps to ensure that the user 108 has ingested some food and decreases the likelihood that the user 108 will become hypoglycemic due to delivery of a partial bolus dose. If the glucose level of the user 108 rises sufficiently, at 1308, the partial insulin bolus dose is delivered to the user 108 by the medicament delivery device 102. After the delivery of the partial bolus, at 1310, a check is performed as to whether the glucose level of the user 108 has peaked or not. A differential and/or slopes of the glucose trajectory may be determined. Once the glucose level begins to trend downward, the glucose level has peaked. If the glucose level has peaked, at 1312, the remainder of the insulin bolus dose is terminated so that it is not delivered to the user. If the glucose level has not peaked, at 1314. an additional portion of the insulin bolus dose is delivered to the user 108. These steps 1310 and 1314 are repeated until the glucose peak is reached or all the insulin bolus dose has been delivered.

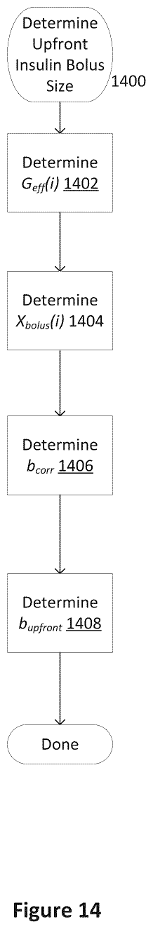

FIG. 14 depicts a flowchart of illustrative steps that may be performed in exemplary embodiments to determine the size of the partial insulin bolus that is delivered initially in 1308. At 1402, the effective glucose for cycle i, G_{eff}(i) is determined. A suitable equation for calculating the effective glucose is:

G_{eff}(i)=G(i)−IOB(i)·CF(i)+2·(G(i)—G(i−2)) (Eq. 2)

where G(i) is the glucose level of the user 108 for cycle i, IOB(i) is the insulin on board for the user 108 for cycle i, and CF(i) is the correction factor for the user for cycle i. The effective glucose value accounts for the insulin action of the IOB(i) and the rate of change in glucose level of the user 108 over the past 15 minutes.

At 1404, X_{bolus} (i.e., the proportion of the bolus that remains after accounting for the correction insulin bolus dose that is needed to bring the blood glucose level of the user 108 to a target level and IOB(i)) are calculated. A suitable equation for determining X_{bolus} is:

$${X}_{bolus} = min ( 1 , \frac{- 40}{target ( i ) - {G}_{eff} ( i )} ) \qquad ( Eq . 3 )$$

where target(i) is the target glucose level for the user 108, and −40 in the numerator is a configurable parameter. If the insulin bolus dose is more than 40 mg/dL below the target, the insulin bolus is reduced because the ratio in Equation 3 will be less than one and the minimum between the ratio and 1 is chosen.

At 1406, the correction insulin bolus dose, b_{corr}, to put the glucose level of the user at target(i) is determined. A suitable equation for calculating b_{corr} is:

$${b}_{corr} = \frac{G ( i ) - target ( i )}{CF ( i )} - IOB ( i ) \qquad ( Eq . 4 )$$

This equation determines how much insulin is needed to reach target(i) given the current glucose level g(i) and IOB(i). That amount of insulin is the dose for the correction bolus, b_{corr} Once b_{corr} is determined, the upfront bolus portion may be determined at 1408 as:

b_{upfront}=X_{bolus}·max(0,b(i)−b_{corr})+min(b_{corr},b(i) (Eq. 5).

Thus, if the correction bolus b_{corr} is larger than the insulin bolus dose b(i) requested by the user 108, the upfront portion of the insulin bolus dose that is delivered is b(i). If b(i) is larger than the correction bolus, the upfront portion of the insulin bolus dose that is delivered is the product of X_{bolus} and b(i) plus b_{corr}.

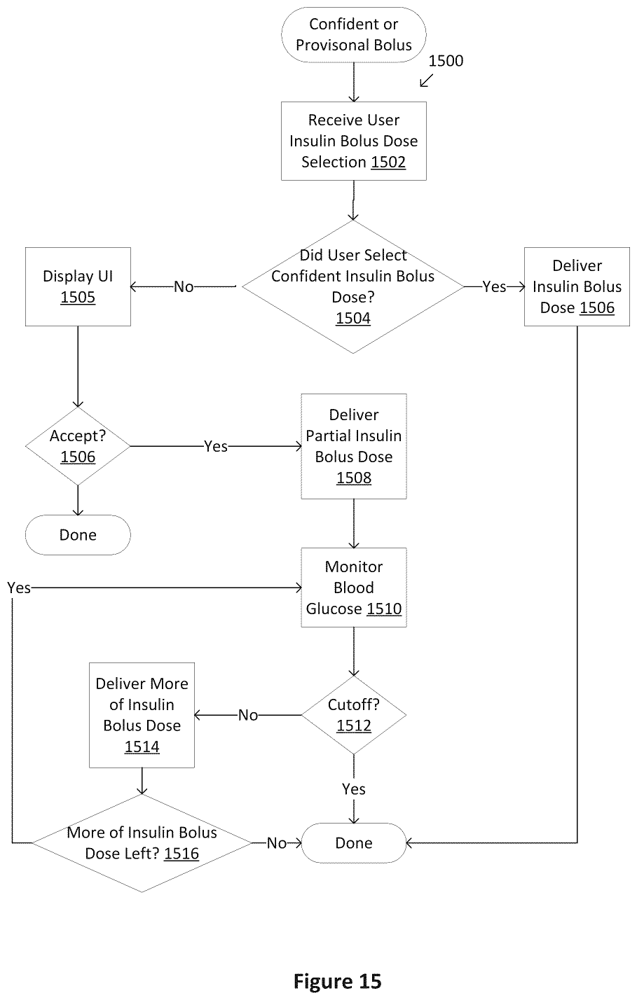

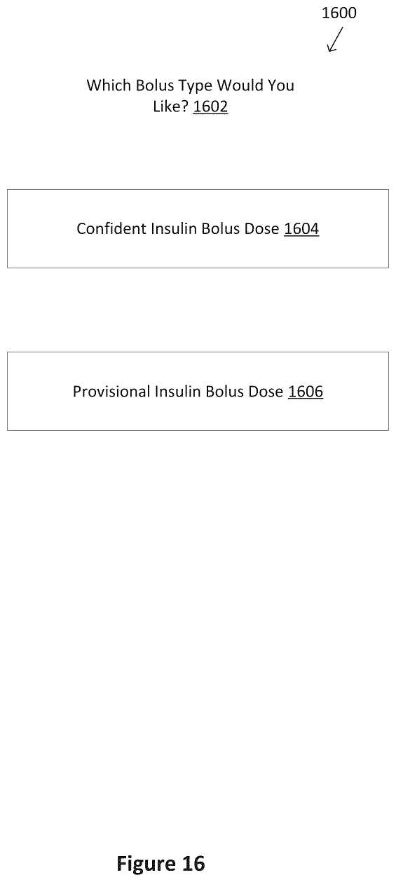

The medicament delivery device 102 may support provisional insulin bolus doses. A provisional insulin bolus dose is one that may be modified based on glucose level trends. In contrast, a confident insulin bolus dose is one that the user 108 is confident in and does not wish for the medicament delivery device 102 to modify the insulin bolus dose. FIG. 15 depicts a flowchart 1500 of illustrative steps that may be performed relative to provisional insulin bolus doses. At 1502, a user's insulin bolus dose selection of a provisional insulin bolus dose or a confident insulin bolus dose is received. A user interface, like user interface 1600 of FIG. 16, may be displayed on the display 127 of the medicament delivery device 102 or on display 140 of the management device 104. The user interface 1600 contains a prompt 1602 for the user 108 to select a button 1604 for a confident insulin bolus dose or a button 1606 for a provisional insulin bolus dose.

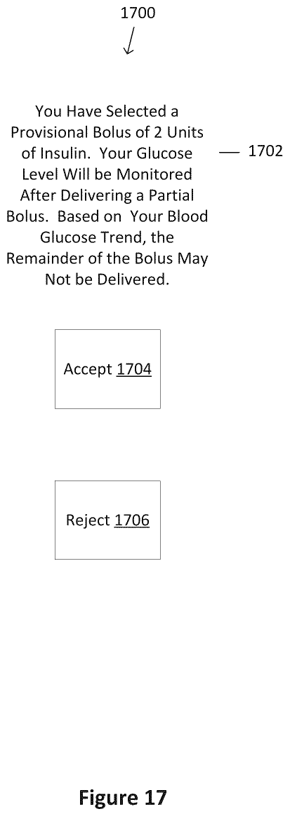

At 1504, a check is made of whether the user 108 selected the confident insulin bolus button 1604. If the confident insulin bolus dose button 1604 is selected, at 1506, the user requested insulin bolus dose is delivered by the medicament delivery device 102. If not and by default the provisional insulin bolus dose button is selected, at 1505, before the delivery of the provisional insulin bolus dose, a user interface 1700 like that shown in FIG. 17, may be shown. The user interface 1700 includes an explanation 1702 that a portion of the insulin bolus dose will be delivered and that the remainder may not be delivered based on glucose level readings of the user 108. At 1506, the user 108 may select the accept button 1704 to deliver the first portion of the provisional insulin bolus dose at 1508, or may select the reject button 1706 to reject the provisional insulin bolus dose so that no insulin bolus dose is delivered. The process may move directly from 1504 to 1508 without an intervening UI and confirmation.

If the partial insulin bolus dose is delivered, at 1510, the blood glucose level of the user is monitored. At 1512, a check is made of the blood glucose level trend of the user 108 to determine whether or not to deliver any more of the insulin bolus dose. At 1514, if the blood glucose trend indicates that more of the insulin bolus dose is warranted, an additional portion of the insulin bolus dose is delivered. If more of the insulin bolus dose is left, as determined at 1516, the process repeats at 1510. If not, the process is complete. If at 1512, the blood glucose level indicates that no more insulin bolus dose is warranted, the remainder of the insulin bolus dose is terminated so as to not be delivered. A message may be output to the user via a UI to indicate that the remainder of the insulin bolus was not delivered and may indicate the amount that was not delivered and that was delivered.

In many instances, a user 108 may receive medicament from more than one delivery device. This poses a challenge in keeping track of how much medicament has been delivered by each device and when. The exemplary embodiments provide a number of approaches for ensuring that at least some of the devices are aware of what other devices have delivered and when.

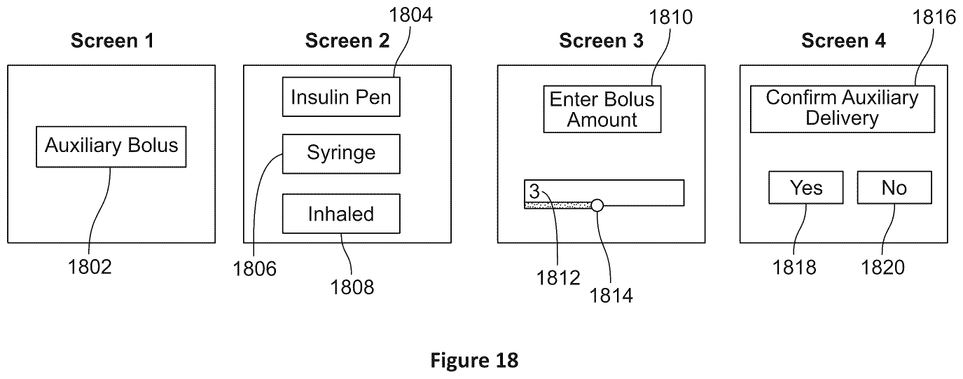

One approach that may be adopted in the exemplary embodiments is to provide the user 108 with a user interface to input medicament deliveries by delivery devices other than the medicament delivery device 102, particularly if such other delivery devices are not smart delivery devices able to output delivered amounts to other devices. FIG. 18 depicts an illustrative user interface screen sequence (labelled as screens 1-4) that may be displayed for the user to enter information regarding an “auxiliary bolus” that was delivered by a device other than the medicament delivery device 102. In screen 1, an auxiliary bolus button 1802 is provided that may be selected to enter information regarding an auxiliary bolus that was delivered by another delivery device. In screen 2, buttons 1804, 1806 and 1808 are displayed for selection of the delivery device that delivered the auxiliary bolus to the user 108. Button 1804 is for an insulin pen, button 1806 is for a syringe, and button 1808 is for an inhaler or other device through which medicament is inhaled. The listing of these devices is intended to be merely illustrative and not limiting. In screen 3, a prompt 1810 prompts the user 108 to enter the dose size of the auxiliary bolus. The user 108 may type a value in box 1812, such as 3 units or may use a slider 1814 to increase or decrease the size specified in the box 1812. Screen 4 contains text 1816 asking the user to confirm that the auxiliary bolus was delivered by selecting “Yes” button 1818 or to reject the auxiliary bolus delivery by selecting the “No” button 1820. Thus, information is gathered via screens 1-4 and the medicament delivery device 102 becomes aware of the gathered information. The information then may be used in calculating bolus sizes, approving or rejecting medicament bolus request and/or adjusting bolus doses.

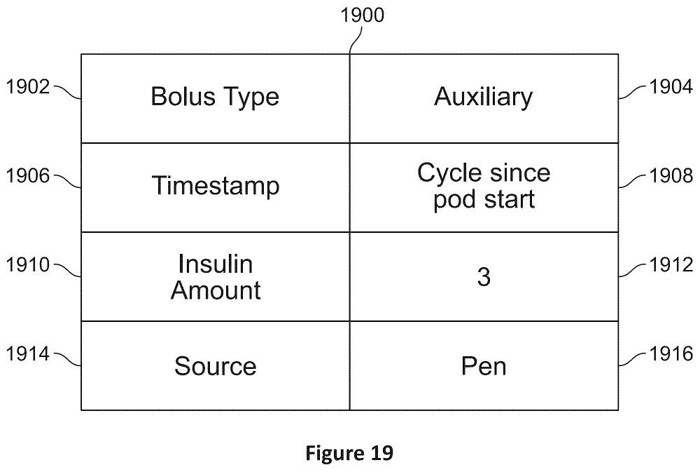

FIG. 19 depicts an illustrative message 1900 that may be sent to the medicament delivery device 102 from another device, such as the management device 104, to provide information regarding the auxiliary bolus. The message 1900 may include a bolus type field 1902, which in this example is “auxiliary” 1904. The message 1900 also may include a timestamp field 1906, which holds a value 1908 that identifies a cycle number since the medicament delivery device started. The message further may include an insulin amount field 1910 that holds a value 1912 of 3 units in this example. The message additionally may include a source field 1914 that holds information 1916 that identifies the type of device that delivered the auxiliary bonus, such as a pen type device.

In some instances, medicament bolus delivery recommendations may originate from multiple sources. For instance, the control application 116 of the medicament delivery device 102 or a bolus calculator of the management device 104 may provide recommendations. These recommendations may clash as to dose, and, depending on latency of communication and up-to-date knowledge of medicament delivery history of the user 108 and other state information that influences medicament bolus recommendations and deliveries, errors in medicament bolus delivery may occur. One approach to addressing these issues that may be adopted in exemplary embodiments is to provide a “broker” that reconciles competing recommendations or requests, or recommendations or requests that are close in time to each other. The broker analyzes the recommendations or requests and ensures that no issues will arise due to other recommendations or requests or a lack of current knowledge before proceeding with a medicament bolus delivery. The broker may be part of the control application 116 on medicament delivery device 102 in some embodiments, but may also be a separate entity in other embodiments.

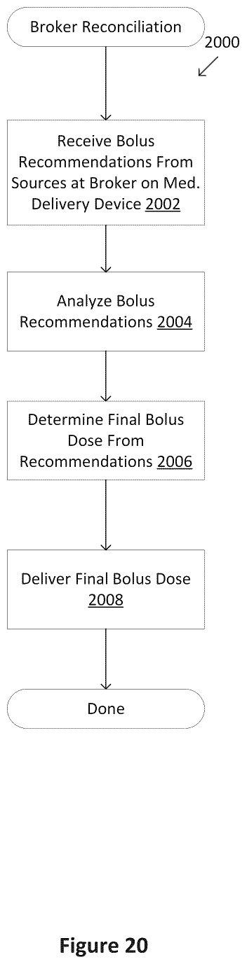

FIG. 20 depicts a flowchart of illustrative steps that may be performed in exemplary embodiments to use the broker to perform the reconciliation among recommendations or requests for medicament bolus delivery. At 2002, the broker receives recommendations or requests from multiple sources on the medicament delivery devices. As was mentioned above, a recommendation or request may originate from the management device 104, such as from a bolus calculator, and another recommendation or request may originate from the medicament delivery device 102. Additionally, in some exemplary embodiments, recommendations or requests may originate from other sources, such as from a smartwatch 130, a fitness monitor 132, or another device 134. At 2004, the broker analyzes the recommendations or requests and decides how to reconcile the recommendations and requests. Based on the decided reconciliation, at 2006, the broker determines a final dose from the recommendations and requests. At 2008, the determined final bolus dose is delivered to the user 108 by the medicament delivery device 102.

Consider the case where the management device 104 issues a recommendation to the medicament delivery device 102 and the medicament delivery device 102 also issues a recommendation. For purposes of this example, it is presumed that the medicament is insulin. The medicament delivery device 102 may calculate the bolus using a formula such as:

$$Bolus Dose = x * TDI + \frac{( B G - SetPoint + Elevation ) ) * TDI}{CorrectionFactor} - max ( IOB , 0 ) \qquad ( Eq . 6 )$$

where SetPoint is the target glucose level, BG is the most recent glucose level reading for the user 108, Elevation is in the range of 0 to 50 mg/dL, and x is a configurable parameter in a range from 3% to 15%.

The management device 104 may calculate the insulin bolus dose as:

$$Bolus Dose = y * TDI + \frac{( B G - SetPoint ) ) * TDI}{CorrectionFactor} - max ( IOB , 0 ) \qquad ( Eq . 7 )$$

where y is a configurable parameter in a range from 3% to 15%.

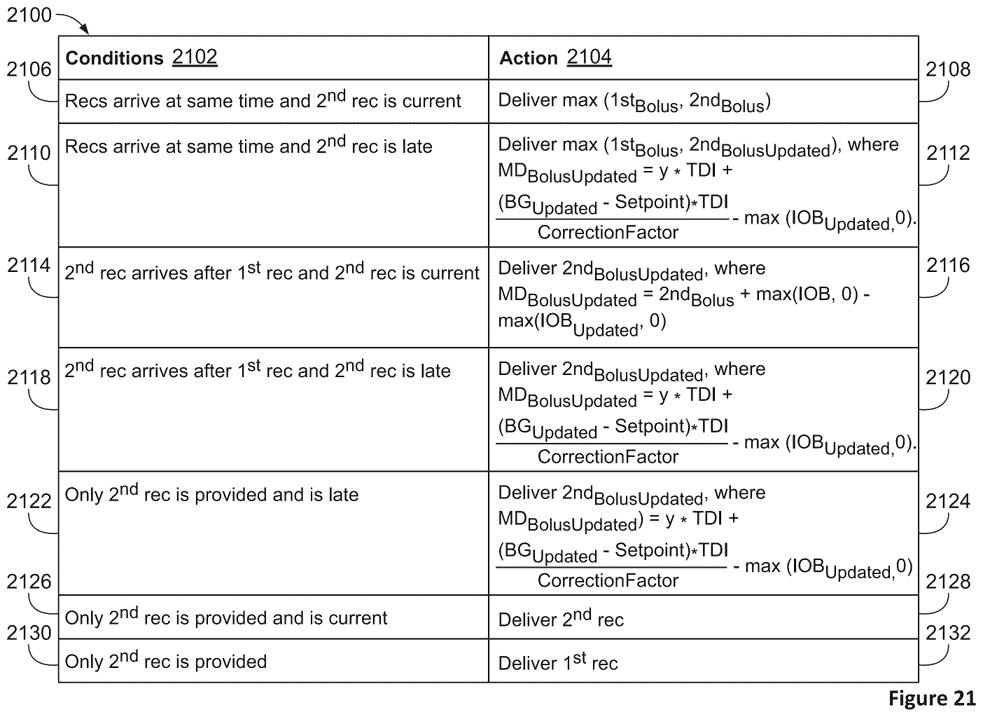

FIG. 21 depicts a table 2100 of conditions 2102 and actions 2104 that are taken to determine the final dose from recommendations at 2006. For purposes of the table 2100, the first recommendation is from the medicament delivery device 102, and the second recommendation is from the management device 104. The conditions 2102 largely concern whether a recommendation is late and when the recommendations that need to be reconciled arrive at the broker. If the conditions are 2106 where the recommendations arrive at the same time and the second recommendation is current, since both recommendations are correct, the larger dose of the two recommendations is delivered. For conditions 2110, where the recommendations arrive at the same time, but the second recommendation is late, the action 2112 is that the bolus dose is the maximum of the bolus recommended by medicament delivery device 102 and an updated recommendation from the management device 104 to make it current (i.e., based on current information for the current cycle). The second recommendation may be updated as follows:

$${MD}_{BolusUpdated} = y * TDI + \frac{( B {G}_{Updates} - SetPoint ) ) * TDI}{CorrectionFactor} - max ( {IOB}_{Updated} , 0 ) \qquad ( Eq . 8 )$$

where BG_{updated} is the most recent glucose level reading for the user 108 and IOB_{updated} is the IOB for the current cycle.

For conditions 2114, where the second recommendation arrives after the first recommendation and the second recommendation is current, the action 2116 is to recalculate and deliver the bolus recommendation from the management device to account for the updated IOB because the insulin bolus for the first recommendation has already been delivered. The updated recommendation may be calculated as:

MD_{BolusUpdated}=MD_{Bolus}+max(IOB,0)−max(IOB_{updated,}O) (Eq. 9)

where MD_{Bolus} is the original recommendation from the management device and IOB_{updated} is the updated IOB value.

For conditions 2118, where the second recommendation arrives after the first recommendation and the second recommendation is late, the action 2120 is to update and deliver the second recommendation to account for the updated IOB and update glucose level reading. The recalculated bolus recommendation from the management device 104 may be determined using Equation 8.

For conditions 2122, where only the second recommendation is provided and is late, the action 2124 is to update and deliver the second recommendation dose from the management device 104. Equation 8 may be used to determine the updated second recommendation.

For conditions 2126, where only the second recommendation is provided and is current, the action 2128 is to deliver the second recommendation. For conditions 2130, where only the first recommendation is provided, the action 2132 is to deliver the first recommendation dose to the user 108.

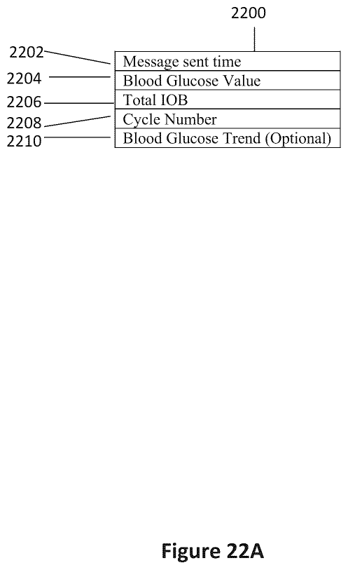

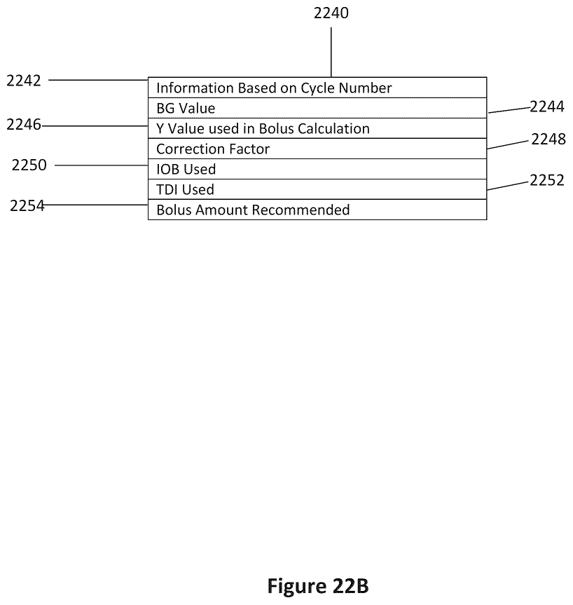

In order to facilitate sharing of information between the medicament delivery device 102 and the management device 104 is the above described process of reconciliation by a broker, the medicament delivery device 102 and management device 104 may exchange messages. FIG. 22A depicts a format of an illustrative message 2200 that may be sent from the medicament delivery device 102 to the management device 104 in order to facilitate the sharing of information. The message 2200 may include a timestamp 2202 of when the message was sent. The message 2200 also may include a blood glucose value of the user 102. The message 2200 may include an indication of the total IOB 2206 per the medicament delivery device 102. The message 2200 also may include a cycle number 2208 and may include information regarding a blood glucose trend 2210. This information in the message 2200 may be used by the management device 104 in determining the bolus dose for the user 108 that is part of a recommendation.

The management device 104 also may send messages to the medicament delivery device 102. FIG. 22B depicts the format of an illustrative message 2240 that may be sent from the management device 104 to the medicament delivery device 102. The message 2240 may contain information that was used in determining a bolus dose for a recommendation from the management device 104. The message 2240 may be used by the medicament delivery device to make final calculations to deliver a correct dose of medicament (e.g., insulin). The message 2240 may contain information particular to the cycle number 2242 and a current blood glucose level value 2244. The message 2240 may contain the value of y 2246 that was used in calculating the bolus dose (see Equation 8). The message 2240 may contain a correction factor value 2248 that was used in determining a bolus dose. Similarly, the message 2240 may contain an IOB value 2250 and a TDI value 2252 that were used in calculating the bolus dose. In addition, the message 2240 may contain the bolus dose that was recommended 2254.

Another solution to address the issue with having multiple medicament bolus recommendation for multiple sources without shared knowledge and without synchronization mechanisms is to have a single actor that is responsible for requests, recommendations and initiating deliveries of medicament. By adopting that approach, exemplary embodiments eliminate synchronization challenges and problems related to knowledge of other actors. The single actor in the exemplary embodiments has complete knowledge of the requests, recommendations and deliveries as the single actor is the only actor.

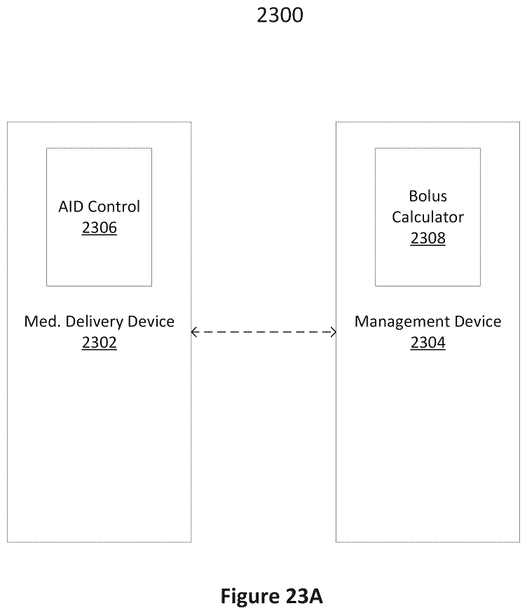

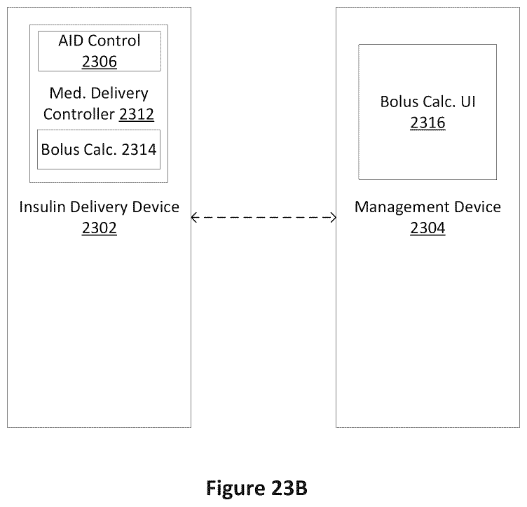

FIG. 23A depicts a conventional approach wherein there is an actor on a medicament delivery device 2302 and another actor on the management device 2304. In this example, the medicament delivery device 102 is a medicament delivery device that includes an automated insulin delivery (AID) control component 2306, such as a control application that oversees and implements the AID algorithm of the medicament delivery device 2302. The AID control component 2306 may calculate an insulin bolus dose based on parameters, such as blood glucose level, JOB, TDI, glucose level target, etc. Here, the “insulin bolus dose” may be termed “microboluses” or “automated basal deliveries” of insulin. The second actor is a bolus calculator 2308 on a management device 2304 that can send user-initiated bolus requests or bolus instructions to the medicament delivery device 2302. The bolus calculator 2308 may include a user interface for interacting with the user to receive input for a user requested insulin bolus. The bolus calculator 2308 may include logic for calculating an insulin bolus based on, for example, current blood glucose level, target glucose level, number of carbohydrates to be ingested in a meal, correction factor, etc. These two actors 2306 and 2308 may separately provide insulin bolus dose recommendations. The two actors 2306 and 2308 are not synchronized and do not necessarily possess complete and current state information regarding blood glucose levels, IOB, and recommendations/bolus deliveries.

FIG. 23B depicts an illustrative arrangement suitable for exemplary embodiments where there is only a single actor, or a single actor that controls actual medicament deliveries. The single actor is the medicament delivery controller 2312 on the medicament delivery device 2302. The medicament delivery controller 2312 contains the AID control component 2306 and the bolus calculator 2314. The medicament delivery controller 2312 is the component that receives recommendations (or requests or instructions) from the AID control component 2306 and also from the bolus calculator 2314, and is the lone component that may trigger an insulin bolus delivery based on the requests or recommendations. The medicament delivery controller 2312 may be part of control application 116 or may be realized in software as computer programming instructions. The user interface of the bolus calculator 2316 may remain on the management device 2304 or another device, like smartwatch 130, fitness monitor 132, or another remote device (and the user interface 231 and calculator 2314 may communicate with each other wirelessly), but the bolus calculator user interface 2316 cannot provide a recommendation or request or instructions that are not first assessed by the medicament delivery controller 2312 before ultimate medicament delivery. Nor can the bolus calculator user interface 2316 initiate an insulin bolus dose delivery, even if the user interface were directly on the medicament delivery device 2302, without first being assessed by a medicament delivery controller 2312, which performs various assessments as outlined above.

In an alternative embodiment, the AID control component 2306 may be resident on a common printed circuit board or may be executed on a common processor with the bolus calculator 2314 to minimize latency and to avoid the problems discussed above. The AID control component 2306 and bolus calculator 2314 may have access to shared state information in some exemplary embodiments. Alternatively, certain synchronization mechanisms may be used to ensure that a medicament bolus is not delivered asynchronously relative to the other actor. Rather, all requests, recommendations, or delivery instructions are assessed and take into account other insulin deliveries, as outlined above.

Software related implementations of the techniques described herein may include, but are not limited to, firmware, application specific software, or any other type of computer readable instructions that may be executed by one or more processors. Hardware related implementations of the techniques described herein may include, but are not limited to, integrated circuits (ICs), application specific ICs (ASICs), field programmable arrays (FPGAs), and/or programmable logic devices (PLDs). In some examples, the techniques described herein, and/or any system or constituent component described herein may be implemented with a processor executing computer readable instructions stored on one or more memory components.

In addition, or alternatively, while the examples may have been described with reference to a closed loop algorithmic implementation, variations of the disclosed examples may be implemented to enable open loop use. The open loop implementations allow for use of different modalities of delivery of insulin such as smart pen, syringe or the like. For example, the disclosed AP application and algorithms may be operable to perform various functions related to open loop operations, such as the generation of prompts requesting the input of information such as weight or age. Similarly, a dosage amount of insulin may be received by the AP application or algorithm from a user via a user interface. Other open-loop actions may also be implemented by adjusting user settings or the like in an AP application or algorithm.

Some examples of the disclosed device may be implemented, for example, using a storage medium, a computer-readable medium, or an article of manufacture which may store an instruction or a set of instructions that, if executed by a machine (i.e., processor or microcontroller), may cause the machine to perform a method and/or operation in accordance with examples of the disclosure. Such a machine may include, for example, any suitable processing platform, computing platform, computing device, processing device, computing system, processing system, computer, processor, or the like, and may be implemented using any suitable combination of hardware and/or software. The computer-readable medium or article may include, for example, any suitable type of memory unit, memory, memory article, memory medium, storage device, storage article, storage medium and/or storage unit, for example, memory (including non-transitory memory), removable or non-removable media, erasable or non-erasable media, writeable or re-writeable media, digital or analog media, hard disk, floppy disk, Compact Disk Read Only Memory (CD-ROM), Compact Disk Recordable (CD-R), Compact Disk Rewriteable (CD-RW), optical disk, magnetic media, magneto-optical media, removable memory cards or disks, various types of Digital Versatile Disk (DVD), a tape, a cassette, or the like. The instructions may include any suitable type of code, such as source code, compiled code, interpreted code, executable code, static code, dynamic code, encrypted code, programming code, and the like, implemented using any suitable high-level, low-level, object-oriented, visual, compiled and/or interpreted programming language. The non-transitory computer readable medium embodied programming code may cause a processor when executing the programming code to perform functions, such as those described herein.

Certain examples of the present disclosure were described above. It is, however, expressly noted that the present disclosure is not limited to those examples, but rather the intention is that additions and modifications to what was expressly described herein are also included within the scope of the disclosed examples. Moreover, it is to be understood that the features of the various examples described herein were not mutually exclusive and may exist in various combinations and permutations, even if such combinations or permutations were not made express herein, without departing from the spirit and scope of the disclosed examples. In fact, variations, modifications, and other implementations of what was described herein will occur to those of ordinary skill in the art without departing from the spirit and the scope of the disclosed examples. As such, the disclosed examples are not to be defined only by the preceding illustrative description.

Program aspects of the technology may be thought of as “products” or “articles of manufacture” typically in the form of executable code and/or associated data that is carried on or embodied in a type of non-transitory, machine readable medium. Storage type media include any or all of the tangible memory of the computers, processors or the like, or associated modules thereof, such as various semiconductor memories, tape drives, disk drives and the like, which may provide non-transitory storage at any time for the software programming. It is emphasized that the Abstract of the Disclosure is provided to allow a reader to quickly ascertain the nature of the technical disclosure. It is submitted with the understanding that it will not be used to interpret or limit the scope or meaning of the claims. In addition, in the foregoing Detailed Description, various features are grouped together in a single example for streamlining the disclosure. This method of disclosure is not to be interpreted as reflecting an intention that the claimed examples require more features than are expressly recited in each claim. Rather, as the following claims reflect, inventive subject matter lies in less than all features of a single disclosed example. Thus, the following claims are hereby incorporated into the Detailed Description, with each claim standing on its own as a separate example. In the appended claims, the terms “including” and “in which” are used as the plain-English equivalents of the respective terms “comprising” and “wherein,” respectively. Moreover, the terms “first,” “second,” “third,” and so forth, are used merely as labels and are not intended to impose numerical requirements on their objects.

The foregoing description of examples has been presented for the purposes of illustration and description. It is not intended to be exhaustive or to limit the present disclosure to the precise forms disclosed. Many modifications and variations are possible in light of this disclosure. It is intended that the scope of the present disclosure be limited not by this detailed description, but rather by the claims appended hereto. Future filed applications claiming priority to this application may claim the disclosed subject matter in a different manner and may generally include any set of one or more limitations as variously disclosed or otherwise demonstrated herein.

What is claimed is:

1. A method performed by a processor of an insulin delivery device, comprising:
monitoring glucose level values for a user with the processor of the insulin delivery device;
receiving an insulin bolus request for delivery of a specified bolus dose of insulin via the insulin delivery device to the user;
with the processor of the insulin delivery device, determining a glucose level trend of the user based on the monitoring;
with the processor of the insulin delivery device, adjusting the specified bolus dose of insulin in the insulin bolus request based on the determined glucose level trend of the user showing a glucose level characteristic; and
delivering an insulin bolus of the adjusted specified bolus dose to the user via the insulin delivery device.

2. The method of claim 1, wherein:
the glucose level characteristic is a substantial rise in glucose level of the user;
only where the checking indicates there is substantial rise in glucose level of the user, performing the adjusting of the specified bolus dose.

3. The method of claim 2, wherein the adjusting comprises determining a corrected insulin bolus dose to compensate for ingestion of a meal by the user.

4. The method of claim 3, wherein the larger of the corrected insulin bolus dose and the specified dose is set as the adjusted specified bolus dose and delivered to the user via the insulin delivery device.

5. The method of claim 2, wherein
where the checking determines that there is no substantial rise in glucose level of the user,
delivering an insulin bolus of the specified bolus dose to the user via the insulin delivery device,
not performing the adjusting of the specified bolus dose, and
not delivering an insulin bolus of the adjusted specified bolus dose to the user via the insulin delivery device.

6. The method of claim 1, further comprising displaying on a display a notification that the adjusted specified bolus dose of insulin was delivered to the user via the insulin delivery device.

7. The method of claim 1, further comprising displaying on the display a confirmation request for confirming delivery of the adjusted specified dose.

8. A method performed by a processor of an insulin delivery device, comprising:
receiving a request for delivery of an insulin bolus of a specified bolus dose to a user by an insulin delivery device;
determining with the processor of the insulin delivery device whether the user likely has eaten within an interval and/or whether the user has already received a bolus of insulin within a time window;
where it is determined that the user likely has eaten within the interval and/or the user has already received a bolus of insulin within the time window, adjusting the specified bolus dose of the requested insulin bolus with the processor of the insulin delivery device to account for the user likely having eaten and/or the user having already received the bolus of insulin in the time window; and
with the processor of the insulin delivery device, causing delivery of the insulin bolus of the adjusted specified bolus dose to the user by the insulin delivery device.

9. The method of claim 8, wherein the request is from an external electronic device.

10. The method of claim 9, wherein the external electronic device is a management device for the insulin delivery device.

11. The method of claim 8, wherein the determining whether the user likely has eaten within an interval comprises analyzing a glucose level trend of the user.

12. The method of claim 11, wherein the analyzing the glucose level trend of the user comprises analyzing the glucose level trend to identify a rise in glucose level associated with ingesting a meal.

13. The method of claim 11, wherein the analyzing the glucose level is performed by at least one machine learning model.

14. The method of claim 13, wherein the analyzing the glucose level is performed by multiple machine learning models.

15. The method of claim 8, wherein it is determined that the user likely has eaten within the interval and wherein the adjusting the dose of the requested insulin bolus comprises increasing the dose of the requested insulin bolus.

16. The method of claim 8, wherein it is determined that the user likely has not eaten within the interval and the adjusting the dose of the requested insulin bolus comprises decreasing the dose of the requested insulin bolus.

17. The method of claim 8, wherein it is determined that the user has already received a bolus of insulin within the time window and the adjusting the dose of the requested insulin bolus comprises decreasing the dose of the requested insulin bolus.

18. The method of claim 8, wherein it is determined that the user is not likely to have eaten within the interval and the adjusting the dose of the requested insulin bolus with the processor of the insulin delivery device comprises adjusting the dose of the requested insulin bolus to zero.

19. The method of claim 8, wherein it is determined that the user has already received a bolus of insulin within the time window and the adjusting the dose of the requested insulin bolus with the processor of the insulin delivery device comprises adjusting the dose of the requested insulin bolus to zero.

20. An insulin delivery system for delivering insulin to a user, comprising:
an insulin delivery device comprising:
a reservoir storing insulin;
a non-transitory computer-readable storage medium for storing computer programming instructions for a single entity that is in control of determining doses and causing delivery of insulin to the user from the insulin delivery device to the user, the single entity including:
a bolus calculator for calculating doses for insulin boluses to be delivered to the user, and
a controller for controlling automated insulin delivery to the user;
a processor for executing the computer programming instructions of the single entity to determine bolus doses and cause delivery of insulin boluses to the user from the insulin delivery device; and
a user interface in communication with the bolus calculator.

21. The insulin delivery system of claim 20, wherein the user interface is on a remote device that is remote from the insulin delivery device.

22. The insulin delivery system of claim 20, wherein the remote device is a smartphone or a smartwatch,
